(19) United States
(12) Patent Application Publication
(10) Pub. No.: US 2004/0115156 A1
(43) Pub. Date: Jun. 17, 2004

(54) Cosmetic composition comprising a fixing polymer and a cationic poly(vinyllactam)

(76) Inventors: Roland De La Mettrie, Le Vesinet (FR); Emanuelle Belli, Asnieres (FR); Laurence Marie, Saint-Denis (FR)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/470,450

(22) PCT Filed: Jan. 24, 2002
(86) PCT No.: PCT/FR02/00292

(30) Foreign Application Priority Data
Jan. 26, 2001 (FR) 01 01112

(51) Int. Cl. A61K 7/06; A61K 7/11

(52) U.S. Cl. 424/70.15

(57) ABSTRACT

The invention concerns novel cosmetic composition for hair care comprising, in a cosmetically acceptable medium: (a) at least a cationic poly(vinyllactam), and (b) at least a fixing polymer selected among anionic, amphoteric, non-ionic fixing agents and their mixtures.

The invention relates to a cosmetic composition, especially a gel or a mousse, comprising at least one fixing polymer and at least one particular cationic polymer. The invention is also directed toward a cosmetic haircare process comprising the application of this composition to the hair, and also toward its use for fixing and/or holding the hairstyle.

Products for fixing and/or holding the hairstyle that are in various forms, in particular in the form of gels or mousses, are known. Although they often allow long-lasting fixing of the hairstyle, they nevertheless have various drawbacks.

In particular, many consumers complain that the gel remains stuck on the hands during application, that it takes a long time to dry on the hair, which may lead to a collapse of the hairstyle if the gel is applied at the end to dry hair, and that it gives the hair an artificial appearance, in particular an unnatural sheen.

Similarly, when the composition is packaged in the form of a mousse, the foaming agents can give the composition a sticky feel. Moreover, the hair occasionally has a dry appearance after applying and drying styling mousses.

The problem posed by the invention is that of providing a cosmetic hair composition, in particular in the form of a gel or a mousse, which is better than the compositions of the prior art, and in particular which leaves the hands clean after it has been applied to the hair, makes possible an application to dry hair without destructuring the hairstyle, dries quickly, holds the shape of the hairstyle strongly and is distributed homogeneously, while at the same time giving the hair good cosmetic properties.

The Applicant has discovered, surprisingly and unexpectedly, that by carefully selecting the constituents to make the cosmetic hair composition, it is possible to solve the problem posed above.

One subject of the invention is a cosmetic composition, characterized in that it comprises, in a cosmetically acceptable medium:

(i) at least one fixing polymer chosen from anionic, amphoteric and nonionic fixing polymers, and mixtures thereof, and

(ii) at least one cationic poly(vinyllactam) polymer comprising:

a) at least one monomer of vinyllactam or alkylvinyllactam type;

b) at least one monomer of structure (I) or (II) below:

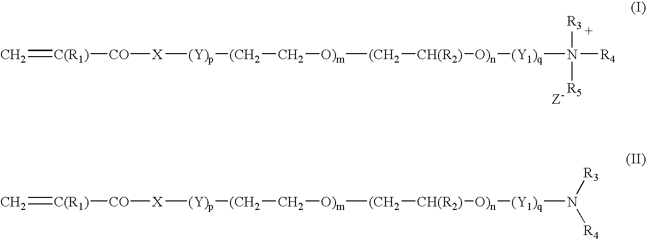

in which:

X denotes an oxygen atom or a radical NR₆,

R₁ and R₆ denote, independently of each other, a hydrogen atom or a linear or branched C₁-C₅ alkyl radical,

R₂ denotes a linear or branched C₁-C₄ alkyl radical,

R₃, R₄ and R₅ denote, independently of each other, a hydrogen atom, a linear or branched C₁-C₃₀ alkyl radical or a radical of formula (III):

—(Y₂)ᵣ—(CH₂—CH(R₇)—O)ₓ—R₈ (III)

Y, Y₁ and Y₂ denote, independently of each other, a linear or branched C₂-C₁₆ alkylene radical,

R₇ denotes a hydrogen atom or a linear or branched C₁-C₄ alkyl radical or a linear or branched C₁-C₄ hydroxyalkyl radical,

R₈ denotes a hydrogen atom or a linear or branched C₁-C₃₀ alkyl radical,

p, q and r denote, independently of each other, either the value 0 or the value 1,

m and n denote, independently of each other, an integer ranging from 0 to 100,

x denotes an integer ranging from 1 to 100,

Z denotes an organic or mineral acid anion,

with the proviso that:

at least one of the substituents R₃, R₄, R₅ or R₈ denotes a linear or branched C₉-C₃₀ alkyl radical,

if m or n is other than zero, then q is equal to 1,

if m or n are equal to zero, then p or q is equal to 0.

Another subject of the invention relates to a cosmetic haircare process, in particular a process for fixing and/or holding the hairstyle using said composition.

Another subject of the invention relates to the use of this composition for holding and/or shaping the hairstyle.

Without wishing to be bound by any theory, it would appear that the advantages provided by the cationic poly(vinyllactams) according to the present invention and as defined hereinbelow are in relation with a behavior of thickening polymers of associative type.

Associative polymers are polymers whose molecules are capable, in the formulation medium, of associating with each other or with molecules of other compounds.

Their chemical structure generally comprises at least one hydrophilic region and at least one hydrophobic region, the hydrophobic region(s) comprising at least one fatty chain.

Cationic Polyvinyllactam Polymers According to the Invention

The cationic poly(vinyllactam) polymers according to the invention comprise:

a) at least one monomer of vinyllactam or alkylvinyllactam type;

b) at least one monomer of structure (I) or (II) below:

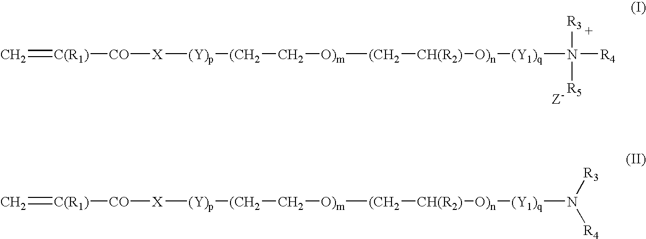

in which:

X denotes an oxygen atom or a radical NR₆,

R₁ and R₆ denote, independently of each other, a hydrogen atom or a linear or branched C₁-C₅ alkyl radical,

R₂ denotes a linear or branched C₁-C₄ alkyl radical,

R₃, R₄ and R₅ denote, independently of each other, a hydrogen atom, a linear or branched C₁-C₃₀ alkyl radical or a radical of formula (III):

—(Y₂)ᵣ—(CH₂—CH(R₇)—O)ₓ—R₈ (III)

Y, Y₁ and Y₂ denote, independently of each other, a linear or branched C₂-C₁₆ alkylene radical,

R₇ denotes a hydrogen atom or a linear or branched C₁-C₄ alkyl radical or a linear or branched C₁-C₄ hydroxyalkyl radical,

R₈ denotes a hydrogen atom or a linear or branched C₁-C₃₀ alkyl radical,

p, q and r denote, independently of each other, either the value 0 or the value 1,

m and n denote, independently of each other, an integer ranging from 0 to 100,

x denotes an integer ranging from 1 to 100,

Z denotes an organic or mineral acid anion,

with the proviso that:

at least one of the substituents R₃, R₄, R₅ or R₈ denotes a linear or branched C₉-C₃₀ alkyl radical,

if m or n is other than zero, then q is equal to

if m or n are equal to zero, then p or q is equal to 0.

The cationic poly(vinyllactam) polymers according to the invention may be crosslinked or noncrosslinked and may also be block polymers.

Preferably, the counterion Z⁻ of the monomers of formula (I) is chosen from halide ions, phosphate ions, the methosulfate ion and the tosylate ion.

Preferably, R₃, R₄ and R₅ denote, independently of each other, a hydrogen atom or a linear or branched C₁-C₃₀ alkyl radical.

More preferably, the monomer b) is a monomer of formula (I) for which, even more preferably, m and n are equal to 0.

The vinyllactam or alkylvinyllactam monomer is preferably a compound of structure (IV):

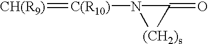

in which:

s denotes an integer ranging from 3 to 6,

R₉ denotes a hydrogen atom or a C₁-C₅ alkyl radical,

R₁₀ denotes a hydrogen atom or a C₁-C₅ alkyl radical, with the proviso that at least one of the radicals R₉ and R₁₀ denotes a hydrogen atom.

Even more preferably, the monomer (IV) is vinylpyrrolidone.

The cationic poly(vinyllactam) polymers according to the invention may also contain one or more additional monomers, preferably cationic or nonionic monomers.

As compounds that are more particularly preferred according to the invention, mention may be made of the following terpolymers comprising at least:

a) one monomer of formula (IV),

b) one monomer of formula (I) in which p=1, q=0, R₃ and R₄ denote, independently of each other, a hydrogen atom or a C₁-C₅ alkyl radical and R₅ denotes a C₉-C₂₄ alkyl radical, and

c) one monomer of formula (II) in which R₃ and R₄ denote, independently of each other, a hydrogen atom or a C₁-C₅ alkyl radical.

Even more preferably, terpolymers comprising, by weight, 40% to 95% of monomer (a), 0.25% to 50% of monomer (b) and 0.1% to 55% of monomer (c) will be used. Such polymers are described in patent application WO 00/68282, the content of which forms an integral part of the invention.

As cationic poly(vinyllactam) polymers according to the invention, vinylpyrrolidone/dimethylaminopropylmethacrylamide/dodecyldimethylmethacrylamidopropylammonium tosylate terpolymers, vinylpyrrolidone/dimethylaminopropylmethacrylamide/cocoyldimethylmethacrylamidopropylammonium tosylate terpolymers, vinylpyrrolidone/dimethylaminopropylmethacrylamide/lauryldimethylmethacrylamidopropylammonium tosylate or chloride terpolymers are used in particular.

The weight-average molecular mass of the cationic poly(vinyllactam) polymers according to the present invention is preferably between 500 and 20 000 000. It is more particularly between 200 000 and 2 000 000 and even more preferably between 400 000 and 800 000.

The cationic poly(vinyllactam) polymers in accordance with the invention are present in the compositions in concentrations ranging from 0.01% to 30% by weight, more preferably from 0.1% to 10% and even more particularly from 0.5% to 2% by weight.

Fixing Polymers

The fixing polymer (b) is chosen from anionic, amphoteric and nonionic fixing polymers, and mixtures thereof. A fixing polymer is a polymer capable of holding and/or fixing the shape of the hairstyle.

These fixing polymers may be used in dissolved form or in the form of a dispersion of solid polymer particles.

The anionic, amphoteric and nonionic fixing polymers (b) that may be used in accordance with the invention are described hereinbelow.

The anionic fixing polymers generally used are polymers containing groups derived from carboxylic acid, sulfonic acid or phosphoric acid and have a molecular weight of approximately between 500 and 5 000 000.

1) The Carboxylic Groups are Provided by Unsaturated Monocarboxylic or Dicarboxylic Acid Monomers such as Those Corresponding to the Formula:

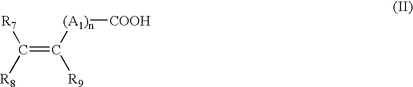

in which n is an integer from 0 to 10, A₁ denotes a methylene group, optionally connected to the carbon atom of the unsaturated group, or to the neighboring methylene group when n is greater than 1, via a hetero atom such as oxygen or sulfur, R₇ denotes a hydrogen atom or a phenyl or benzyl group, R₈ denotes a hydrogen atom or a lower alkyl or carboxyl group, R₉ denotes a hydrogen atom, a lower alkyl group or a —CH₂—COOH, phenyl or benzyl group.

In the abovementioned formula, a lower alkyl radical preferably denotes a group having 1 to 4 carbon atoms and in particular methyl and ethyl.

The anionic fixing polymers containing carboxylic groups that are preferred according to the invention are:

A) acrylic or methacrylic acid homo-or copolymers, or salts thereof and in particular the products sold under the names Versicol E or K by the company Allied Colloid and Ultrahold by the company BASF. The copolymers of acrylic acid and of acrylamide sold in the form of their sodium salt under the names Reten 421, 423 or 425 by the company Hercules, the sodium salts of polyhydroxycarboxylic acids.

B) copolymers of acrylic or methacrylic acids with a monoethylenic monomer such as ethylene, styrene, vinyl esters, acrylic or methacrylic acid esters, optionally grafted onto a polyalkylene glycol such as polyethylene glycol and optionally crosslinked. Such polymers are described in particular in French patent FR-A-1 222 944 and German patent application DE-A-2 330 956, the copolymers of this type containing an optionally N-alkylated and/or hydroxyalkylated acrylamide unit in their chain as described in particular in Luxembourg patent applications 75370 and 75371 or sold under the name Quadramer by the company American Cyanamid. Mention may also be made of copolymers of acrylic acid and of C₁-C₄ alkyl methacrylate and terpolymers of vinylpyrrolidone, of acrylic acid and of methacrylate of C₁-C₂₀ alkyl, for example of lauryl, such as the product sold by the company ISP under the name Acrylidone LM and methacrylic acid/ethyl acrylate/tert-butyl acrylate terpolymers such as the product sold under the name Luvimer 100 P by the company BASF.

C) copolymers derived from crotonic acid such as those containing vinyl acetate or propionate units in their chain and optionally other monomers such as allylic esters or methallylic esters, vinyl ether or vinyl ester of a linear or branched saturated carboxylic acid with a long hydrocarbon chain such as those containing at least 5 carbon atoms, it being possible for these polymers optionally to be grafted and crosslinked, or alternatively a vinyl, allylic or methallylic ester of an α- or β-cyclic carboxylic acid. Such polymers are described, inter alia, in French patents FR-A-1 222 944, FR-A-1 580 545, FR-A-2 265 782, FR-A-2 265 781, FR-A-1 564 110 and FR-A-2 439 798. Commercial products falling into this class are the resins 28-29-30, 26-13-14 and 28-13-10 sold by the company National Starch.

D) copolymers derived from C₄-C₈ monounsaturated carboxylic acids or anhydrides chosen from:

copolymers comprising (i) one or more maleic, fumaric or itaconic acids or anhydrides and

(ii) at least one monomer chosen from vinyl esters, vinyl ethers, vinyl halides, phenylvinyl derivatives, acrylic acid and its esters, the anhydride functions of these copolymers optionally being monoesterified or monoamidated. Such polymers are described in particular in U.S. Pat. Nos. 2,047,398, 2,723,248 and 2,102,113 and GB patent GB-A-0 839 805 and in particular those sold under the names Gantrez AN or ES by the company ISP,

copolymers comprising (i) one or more maleic, citraconic or itaconic anhydrides and (ii) one or more monomers chosen from allylic or methallylic esters optionally containing one or more acrylamide, methacrylamide, α-olefin, acrylic or methacrylic ester, acrylic or methacrylic acid or vinylpyrrolidone groups in their chain,

the anhydride functions of these copolymers optionally being monoesterified or monoamidated.

These polymers are described, for example, in French patents FR-A-2 350 384 and FR-A-2 357 241 by the Applicant.

E) polyacrylamides containing carboxylate groups.

The polymers comprising sulfonic groups are polymers containing vinylsulfonic, styrenesulfonic, naphthalenesulfonic or acrylamidoalkylsulfonic units.

These polymers can be chosen in particular from:

polyvinylsulfonic acid salts having a molecular weight of approximately between 1 000 and 100 000, as well as the copolymers with an unsaturated comonomer such as acrylic or methacrylic acids and their esters, as well as acrylamide or its derivatives, vinyl ethers and vinylpyrrolidone;

polystyrenesulfonic acid salts, the sodium salts having a molecular weight of about 500 000 and about 100 000, which are sold respectively under the names Flexan 500 and Flexan 130 by National Starch. These compounds are described in patent FR-A-2 198 719;

polyacrylamidesulfonic acid salts, those mentioned in U.S. Pat. No. 4,128,631 and more particularly polyacrylamidoethylpropanesulfonic acid sold under the name Cosmedia Polymer HSP 1180 by Henkel.

According to the invention, the anionic fixing polymers are preferably chosen from acrylic acid copolymers, such as the acrylic acid/ethyl acrylate/N-tert-butylacrylamide terpolymer sold under the name Ultrahold Strong by the company BASF, copolymers derived from crotonic acid, such as vinyl acetate/vinyl tert-butylbenzoate/crotonic acid terpolymers and the crotonic acid/vinyl acetate/vinyl neododecanoate terpolymers sold under the name Resin 28-29-30 by the company National Starch, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymer sold under the name Gantrez ES 425 by the company ISP, the copolymers of methacrylic acid and of methyl methacrylate sold under the name Eudragit L by the company Rohm Pharma, the copolymer of methacrylic acid and of ethyl acrylate sold under the name Luvimer MAEX or MAE by the company BASF and the vinyl acetate/crotonic acid copolymer sold under the name Luviset CA 66 by the company BASF and the vinyl acetate/crotonic acid copolymer grafted with polyethylene glycol sold under the name Aristoflex A by the company BASF.

The anionic fixing polymers which are most particularly preferred are chosen from the methyl vinyl ether/monoesterified maleic anhydride copolymer sold under the name Gantrez ES 425 by the company ISP, the acrylic acid/ethyl acrylate/N-tert-butylacrylamide terpolymer sold under the name Ultrahold Strong by the company BASF, the copolymers of methacrylic acid and of methyl methacrylate sold under the name Eudragit L by the company Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, the copolymer of methacrylic acid and of ethyl acrylate sold under the name Luvimer MAEX or MAE by the company BASF and the vinylpyrrolidone/acrylic acid/lauryl methacrylate terpolymer sold under the name Acrylidone LM by the company ISP.

The amphoteric fixing polymers which can be used in accordance with the invention can be chosen from polymers containing units B and C distributed randomly in the polymer chain, in which B denotes a unit derived from a monomer containing at least one basic nitrogen atom and C denotes a unit derived from an acid monomer containing one or more carboxylic or sulfonic groups, or alternatively B and C can denote groups derived from carboxybetaine or sulfobetaine zwitterionic monomers;

B and C can also denote a cationic polymer chain containing primary, secondary, tertiary or quaternary amine groups, in which at least one of the amine groups bears a carboxylic or sulfonic group connected via a hydrocarbon radical or alternatively B and C form part of a chain of a polymer containing an α,β-dicarboxylic ethylene unit in which one of the carboxylic groups has been made to react with a polyamine containing one or more primary or secondary amine groups.

The amphoteric fixing polymers corresponding to the definition given above which are more particularly preferred are chosen from the following polymers:

(1) polymers resulting from the copolymerization of a monomer derived from a vinyl compound bearing a carboxylic group such as, more particularly, acrylic acid, methacrylic acid, maleic acid, α-chloroacrylic acid, and a basic monomer derived from a substituted vinyl compound containing at least one basic atom, such as, more particularly, dialkylaminoalkyl methacrylate and acrylate, dialkylaminoalkylmethacrylamides and-acrylamides. Such compounds are described in U.S. Pat. No. 3,836,537.

(2) polymers containing units derived from:

a) at least one monomer chosen from acrylamides and methacrylamides substituted on the nitrogen with an alkyl radical,

b) at least one acidic comonomer containing one or more reactive carboxylic groups, and

c) at least one basic comonomer such as esters containing primary, secondary, tertiary and quaternary amine substituents of acrylic and methacrylic acids and the product of quaternization of dimethylaminoethyl methacrylate with dimethyl or diethyl sulfate.

The N-substituted acrylamides or methacrylamides which are more particularly preferred according to the invention are groups in which the alkyl radicals contain from 2 to 12 carbon atoms and more particularly N-ethylacrylamide, N-tert-butyl-acrylamide, N-tert-octylacrylamide, N-octylacrylamide, N-decylacrylamide, N-dodecylacrylamide and the corresponding methacrylamides.

The acidic comonomers are chosen more particularly from acrylic acid, methacrylic acid, crotonic acid, itaconic acid, maleic acid and fumaric acid and alkyl monoesters, having 1 to 4 carbon atoms, of maleic or fumaric acids or anhydrides.

The preferred basic comonomers are aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer, such as the products sold under the name Amphomer or Lovocryl 47 by the company National Starch, are particularly used.

(3) crosslinked and alkylated polyamino amides partially or totally derived from polyamino amides of general formula III:

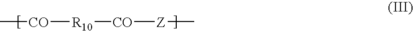

in which R₁₀ represents a divalent radical derived from a saturated dicarboxylic acid, a mono-or dicarboxylic aliphatic acid containing an ethylenic double bond, an ester of a lower alkanol, having 1 to 6 carbon atoms, of these acids, or a radical derived from the addition of any one of said acids to a bis(primary) or bis(secondary) amine, and Z denotes a bis(primary), mono-or bis(secondary) polyalkylene-polyamine radical and preferably represents:

a) in proportions of from 60 to 100 mol %, the radical IV

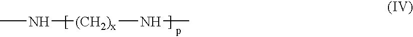

where x=2 and p=2 or 3, or alternatively x=3 and p=2

this radical being derived from diethylenetriamine, from triethylenetetraamine or from dipropylenetriamine;

b) in proportions of from 0 to 40 mol %, the radical (IV) above in which x=2 and p=1 and which is derived from ethylenediamine, or the radical derived from piperazine:

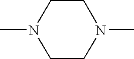

c) in proportions of from 0 to 20 mol %, the —NH(CH₂)₆—NH— radical derived from hexamethylenediamine, these polyamino amines being crosslinked by addition of a difunctional crosslinking agent chosen from epihalohydrins, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

The saturated carboxylic acids are preferably chosen from acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The alkane sultones used in the alkylation are preferably propane sultone or butane sultone, the salts of the alkylating agents are preferably the sodium or potassium salts.

(4) polymers containing zwitterionic units of formula V:

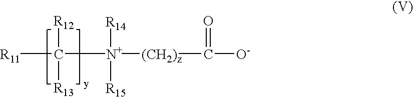

in which R₁₁ denotes a polymerizable unsaturated group such as an acrylate, methacrylate, acrylamide or methacrylamide group, y and z represent an integer from 1 to 3, R₁₂ and R₁₃ represent a hydrogen atom, methyl, ethyl or propyl, R₁₄ and R₁₅ represent a hydrogen atom or an alkyl radical such that the sum of the carbon atoms in R₁₄ and R₁₅ does not exceed 10.

The polymers comprising such units can also contain units derived from nonzwitterionic monomers such as dimethyl or diethylaminoethyl acrylate or methacrylate or alkyl acrylates or methacrylates, acrylamides or methacrylamides or vinyl acetate.

By way of example, mention may be made of the copolymer of methyl methacrylate/methyl dimethylcarboxymethylammonioethylmethacrylate such as the product sold under the name Diaformer Z301 by the company Sandoz.

(5) polymers derived from chitosan containing monomer units corresponding to the following formulae:

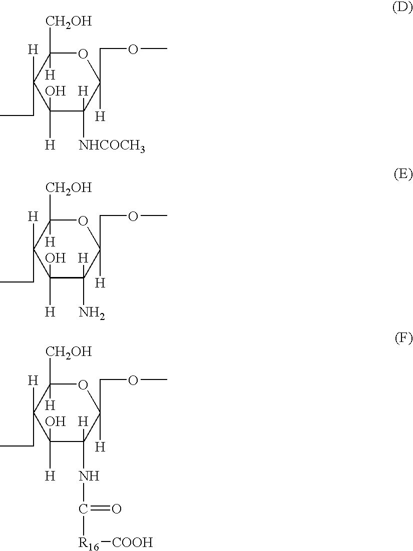

the unit D being present in proportions of between 0 and 30%, the unit E in proportions of between 5 and 50% and the unit F in proportions of between 30 and 90%, it being understood that, in this unit F, R₁₆ represents a radical of formula:

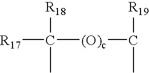

in which, if q=0, R₁₇, R₁₈ and R₁₉, which may be identical or different, each represent a hydrogen atom, a methyl, hydroxyl, acetoxy or amino residue, a monoalkylamine residue or a dialkylamine residue which are optionally interrupted by one or more nitrogen atoms and/or optionally substituted with one or more amine, hydroxyl, carboxyl, alkylthio or sulfonic groups, an alkylthio residue in which the alkyl group bears an amino residue, at least one of the radicals R₁₇, R₁₈ and R₁₉ being, in this case, a hydrogen atom;

or, if q=1, R₁₇, R18 and R₁₉ each represent a hydrogen atom, as well as the salts formed by these compounds with bases or acids.

(6) polymers derived from the N-carboxy-alkylation of chitosan, such as N-carboxymethylchitosan or N-carboxybutylchitosan sold under the name “Evalsan” by the company Jan Dekker.

(7) polymers corresponding to the general formula (VI) which are described, for example, in French patent FR-A-1 400 366:

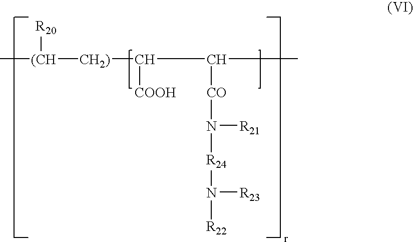

in which R₂₀ represents a hydrogen atom, a CH₃O, CH₃CH₂O or phenyl radical, R₂₁ denotes hydrogen or a lower alkyl radical such as methyl or ethyl, R₂₂ denotes hydrogen or a lower alkyl radical such as methyl or ethyl, R₂₃ denotes a lower alkyl radical such as methyl or ethyl or a radical corresponding to the formula: —R₂₄—N(R₂₂)₂, R₂₄ representing a —CH₂—CH₂—, —CH₂—CH₂—CH₂— or —CH₂—CH(CH₃)— group, R₂₂ having the meanings mentioned above,

as well as the higher homologs of these radicals and containing up to 6 carbon atoms.

(8) amphoteric polymers of the type —D—X—D—X— chosen from:

a) polymers obtained by the action of chloroacetic acid or sodium chloroacetate on compounds containing at least one unit of formula:

—D—X—D—X—D— (VII)

where D denotes a radical

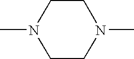

and X denotes the symbol E or E′, E or E′, which may be identical or different, denotes a divalent radical which is an alkylene radical containing a straight or branched chain containing up to 7 carbon atoms in the main chain, which is unsubstituted or substituted with hydroxyl groups and which can contain, in addition to the oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups.

b) polymers of formula:

—D—X—D—X— (VII′)

where D denotes a radical

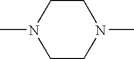

and X denotes the symbol E or E′ and at least once E′; E having the meaning given above and E′ is a divalent radical which is an alkylene radical with a straight or branched chain having up to 7 carbon atoms in the main chain, which is unsubstituted or substituted with one or more hydroxyl radicals and containing one or more nitrogen atoms, the nitrogen atom being substituted with an alkyl chain which is optionally interrupted by an oxygen atom and necessarily containing one or more carboxyl functions or one or more hydroxyl functions and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylamino-propylamine or by semiesterification with an N,N-dialkanolamine. These copolymers can also contain other vinyl comonomers such as vinylcaprolactam.

The amphoteric fixing polymers which are particularly preferred according to the invention are those of family (3), such as the copolymers whose CTFA name is octylacrylamide/acrylates/butylaminoethyl methacrylate copolymer, such as the products sold under the names Amphomer, Amhomer LV 71 or Lovocryl 47 by the company National Starch and those of family (4) such as the copolymer of methyl methacrylate/methyl dimethylcarboxymethylammonioethylmethacrylate, sold, for example, under the name Diaformer Z301 by the company Sandoz.

The nonionic fixing polymers that can be used according to the present invention are chosen, for example, from:

vinylpyrrolidone homopolymers;

copolymers of vinylpyrrolidone and of vinyl acetate;

polyalkyloxazolines such as the polyethyloxazolines sold by the company Dow Chemical under the names Peox 50 000, Peox 200 000 and Peox 500 000;

vinyl acetate homopolymers, such as the product sold under the name Appretan EM by the company Hoechst, or the product sold under the name Rhodopas A 012 by the company Rhône-Poulenc;

copolymers of vinyl acetate and of acrylic ester; such as the product sold under the name Rhodopas AD 310 by Rhêne-Poulenc;

copolymers of vinyl acetate and of ethylene, such as the product sold under the name Appretan TV by the company Hoechst;

copolymers of vinyl acetate and of maleic ester, for example of dibutyl maleate, such as the product sold under the name Appretan MB Extra by the company Hoechst;

copolymers of polyethylene and of maleic anhydride;

alkyl acrylate homopolymers and alkyl methacrylate homopolymers, such as the product sold under the name Micropearl RQ 750 by the company Matsumoto or the product sold under the name Luhydran A 848 S by the company BASF;

acrylic ester copolymers such as, for example, copolymers of alkyl acrylates and of alkyl methacrylates, such as the products sold by the company Rohm & Haas under the names Primal AC-261 K and Eudragit NE 30 D, by the company BASF under the names Acronal 601, Luhydran LR 8833 or 8845, and by the company Hoechst under the names Appretan N 9213 or N 9212;

copolymers of acrylonitrile and of a nonionic monomer chosen, for example, from butadiene and alkyl (meth)acrylates; mention may be made of the products sold under the names Nipol LX 531 B by the company Nippon Zeon or those sold under the name CJ 0601 B by the company Rohm & Haas;

polyurethanes, such as the products sold under the names Acrysol RM 1020 or Acrysol RM 2020 by the company Rohm & Haas, and the products Uraflex XP 401 UZ and Uraflex XP 402 UZ by the company DSM Resins;

copolymers of alkyl acrylate and of urethane, such as the product 8538-33 by the company National Starch;

polyamides, such as the product Estapor LO 11 sold by the company Rhône-Poulenc.

The alkyl radicals of the nonionic polymers have from 1 to 6 carbon atoms except where otherwise mentioned.

The nonionic polymers that are most particularly suitable for preparing the compositions in accordance with the invention are those chosen from:

vinyllactam copolymers such as copolymers of vinylpyrrolidone and of vinyl acetate and vinylpyrrolidone/vinyl acetate/vinyl propionate copolymers

the polyvinylcaprolactam Luviskol Plus (BASF)

vinyl acetate homopolymers such as Appretan EM (Hoechst) or Rhodopas A 012 (Rhône-Poulenc)

polyalkyloxazolines such as Peox 50 000 and Peox 500 000 (Dow Chemical)

copolymers of vinyl acetate and of acrylic ester such as Rhodopas AD 310 (Rhône-Poulenc)

copolymers of vinyl acetate and of ethylene, such as Appretan TV (Hoechst)

copolymers of vinyl acetate and of maleic ester, such as Appretan MB Extra (Hoechst)

alkyl acrylate homopolymers and alkyl methacrylate homopolymers, such as Luhydran A 848 S (BASF)

acrylic ester copolymers such as Primal AC-261 K (Rohm & Haas), Acronal 601 (BASF) or Appretan N 9213 (Hoechst)

copolymers of acrylonitrile and of a nonionic monomer such as CJ 0601 B (Rohm & Haas)

polyurethanes such as Acrysol RM 1020 or Acrysol RM 2020 (Rohm & Haas)

copolymers of alkyl acrylate and of urethane, such as 8538-33 (National Starch)

polyamides such as Estapor LO 11 (Rhône-Poulenc).

According to the invention, it is also possible to use fixing polymers of grafted silicone type comprising a polysiloxane portion and a portion consisting of a nonsilicone organic chain, one of the two portions constituting the main chain of the polymer, the other being grafted onto said main chain. These polymers are described, for example, in patent applications EP-A-0 412 704, EP-A-0 412 707, EP-A-0 640 105 and WO 95/00578, EP-A-0 582 152 and WO 93/23009 and U.S. Pat. Nos. 4,693,935, 4,728,571 and 4,972,037. These polymers are preferably anionic or nonionic.

Such polymers are, for example, copolymers that can be obtained by radical polymerization from the monomer mixture consisting of:

a) 50 to 90% by weight of tert-butyl acrylate;

b) 0 to 40% by weight of acrylic acid;

c) 5 to 40% by weight of silicone macromer of formula:

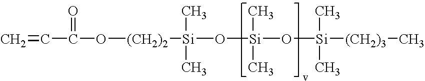

with v being a number from 5 to 700; the weight percentages being calculated relative to the total weight of the monomers.

Other examples of grafted silicone polymers are, in particular, polydimethylsiloxanes (PDMSs) onto which are grafted, via a thiopropylene-type connecting chain, mixed polymer units of the poly(meth)acrylic acid type and of the polyalkyl (meth)acrylate type and polydimethylsiloxanes (PDMSs) onto which are grafted, via a thiopropylene-type connecting chain, polymer units of the polyisobutyl (meth)acrylate type.

Functionalized or nonfunctionalized, silicone or nonsilicone polyurethanes may also be used as fixing polymers. As a polyurethane that may be used in the context of the invention, mention may be made of the product sold under the name Luviset Pur by BASF.

The polyurethanes particularly intended by the present invention are those disclosed in patents EP-A-0 751 162, EP-A-0 637 600, FR-A-2 743 297 and EP-A-0 648 485, of which the Applicant is the Proprietor, and patents EP-A-0 656 021 or WO 94/03510 from the company BASF and EP-A-0 619 111 from the company National Starch.

Advantageously, the fixing polymer is present in the composition in accordance with the invention at a relative weight concentration of between 0.1% and 10% and preferably between 0.5% and 5%.

The cosmetically acceptable medium preferably consists of water or of one or more cosmetically acceptable solvents such as alcohols or water/solvent(s) mixtures, these solvents preferably being C₁-C₄ alcohols.

Among these alcohols, mention may be made of ethanol and isopropanol. Ethanol is particularly preferred.

The composition of the invention can also contain at least one additive chosen from anionic, cationic, nonionic and amphoteric surfactants, fragrances, screening agents, preserving agents, proteins, vitamins, provitamins, polymers other than those of the invention, plant, mineral or synthetic oils, polyols, for instance glycols or glycerol, silicones, fatty alcohols and any other additive conventionally used in cosmetic compositions.

Needless to say, a person skilled in the art will take care to select the optional compound(s) to be added to the composition according to the invention such that the advantageous properties intrinsically associated with the composition in accordance with the invention are not, or are not substantially, adversely affected by the envisaged addition.

The compositions in accordance with the invention may be applied to the skin, the nails, the lips, the hair, the eyebrows and the eyelashes.

The compositions in accordance with the invention are particularly suitable for dry or wet hair, as styling products.

The invention will be illustrated more fully with the aid of the nonlimiting examples that follow.

All the percentages are relative percentages by weight relative to the total weight of the composition, and a.m. means active material.

### EXAMPLES

The compositions below in accordance with the invention are prepared.

Example 1: propellant-free mousse (Airspray F2 pump-
dispenser bottle)

| | |
|---|---|
| Polymer 1 | 2.5% |
| PVP/VA | 1% |
| Glycerol | 0.5% |
| Preserving agents, fragrance | |
| Water | qs 100% |

Example 2: aerosol mousse

| | |
|---|---|
| Polymer 1 | 2.5% |
| PVP | 1% |
| Aerogaz 3.2N (Atochem) | 6% |
| Preserving agents, fragrance | |
| Water | qs 100% |

Example 3: gel

| | |
|---|---|
| Polymer 1 | 2.5% |
| PVP/VA | 0.5% |
| Jaguar HP 105 | 1% |
| Preserving agents, fragrance | |
| Water | qs 100% |

Polymer 1 is a vinylpyrrolidone/dimethylaminopropylmethacrylamide/lauryldimethylmethacrylamidoammonium chloride terpolymer sold by the company ISP under the reference Polymer ACP-1234.

1. A cosmetic composition, characterized in that it comprises, in a cosmetically acceptable medium:
(i) at least one fixing polymer chosen from anionic, amphoteric and nonionic fixing polymers, and mixtures thereof, and
(ii) at least one cationic poly(vinyllactam) polymer comprising:
a) at least one monomer of vinyllactam or alkylvinyllactam type;
b) at least one monomer of structure (I) or (II) below: 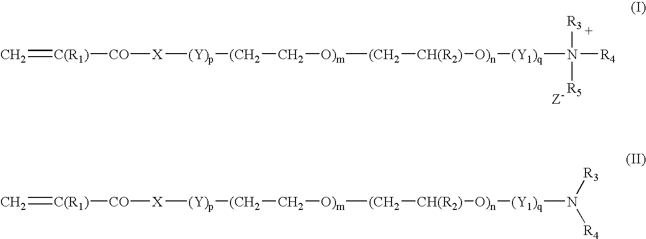
in which:
X denotes an oxygen atom or a radical NR₆,
R₁ and R₆ denote, independently of each other, a hydrogen atom or a linear or branched C₁-C₅ alkyl radical,
R₂ denotes a linear or branched C₁-C₄ alkyl radical,
R₃, R₄ and R₅ denote, independently of each other, a hydrogen atom, a linear or branched C₁-C₃₀ alkyl radical or a radical of formula (III):
—(Y₂)ᵣ—(CH₂—CH(R₇)—O)ₓ—R₈ (III)
Y, Y₁ and Y₂ denote, independently of each other, a linear or branched C₂-C₁₆ alkylene radical,
R₇ denotes a hydrogen atom or a linear or branched C₁-C₄ alkyl radical or a linear or branched C₁-C₄ hydroxyalkyl radical,
R₈ denotes a hydrogen atom or a linear or branched C₁-C₃₀ alkyl radical,
p, q and r denote, independently of each other, either the value 0 or the value 1,
m and n denote, independently of each other, an integer ranging from 0 to 100,
x denotes an integer ranging from 1 to 100,
Z denotes an organic or mineral acid anion,
with the proviso that:
at least one of the substituents R₃, R₄, R₅ or R₈ denotes a linear or branched C₉-C₃₀ alkyl radical,
if m or n is other than zero, then q is equal to 1,
if m or n are equal to zero, then p or q is equal to 0.

2. The composition as claimed in claim 1, characterized in that the vinyllactam or alkylvinyllactam monomer is a compound of structure (IV): 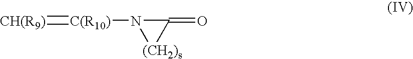
in which:
s denotes an integer ranging from 3 to 6,
R₉ denotes a hydrogen atom or a C₁-C₅ alkyl radical,
R₁₀ denotes a hydrogen atom or a C₁-C₅ alkyl radical,
with the proviso that at least one of the radicals R₉ and R₁₀ denotes a hydrogen atom.

3. The composition as claimed in claim 2, characterized in that the monomer of formula (IV) is vinylpyrrolidone.

4. The composition as claimed in any one of the preceding claims, characterized in that, in formula (I) or (II), the radicals R₃, R₄ and R₅ denote, independently of each other, a hydrogen atom or a linear or branched C₁-C₃₀ alkyl radical.

5. The composition as claimed in any one of the preceding claims, characterized in that the monomer b) is a monomer of formula (I).

6. The composition as claimed in claim 5, characterized in that, in formula (I), m and n are equal to zero.

7. The composition as claimed in any one of the preceding claims, characterized in that the counterion Z⁻ of the monomers of formula (I) is chosen from halide ions, phosphate ions, the methosulfate ion and the tosylate ion.

8. The composition as claimed in any one of claims 1 to 7, characterized in that the cationic poly(vinyllactam) polymer(s) contain(s) one or more additional cationic or nonionic monomers.

9. The composition as claimed in claim 8, characterized in that the cationic poly(vinyllactam) is a terpolymer comprising:
a) a monomer of formula (IV),
b) a monomer of formula (I) in which p=1, q=0, R₃ and R₄ denote, independently of each other, a hydrogen atom or a C₁-C₅ alkyl radical and R₅ denotes a C₉-C₂₄ alkyl radical, and
c) a monomer of formula (II) in which R₃ and R₄ denote, independently of each other, a hydrogen atom or a C₁-C₅ alkyl radical.

10. The composition as claimed in claim 9, characterized in that the terpolymer comprises, by weight, 40% to 95% of monomer (a), 0.25% to 50% of monomer (b) and 0.1% to 55% of monomer (c).

11. The composition as claimed in any one of the preceding claims, characterized in that the cationic poly(vinyllactams) are chosen from vinylpyrrolidone/dimethylaminopropylmethacrylamide/dodecyldimethylmethacrylamidopropylammonium tosylate terpolymers, vinylpyrrolidone/dimethylaminopropylmethacrylamide/cocoyldimethylmethacrylamidopropylammonium tosylate terpolymers, vinylpyrrolidone/dimethylaminopropylmethacrylamide/lauryldimethylmethacrylamidopropylammonium tosylate or chloride terpolymers.

12. The composition as claimed in any one of the preceding claims, characterized in that the weight-average molecular mass of the cationic poly(vinyllactams) is between 500 and 20 000 000, preferably between 200 000 and 2 000 000 and even more preferably between 400 000 and 800 000.

13. The composition as claimed in any one of the preceding claims, characterized in that the cationic poly(vinyllactam(s)) is (are) used in an amount ranging from 0.01% to 30% by weight relative to the total weight of the composition.

14. The composition as claimed in claim 13, characterized in that the cationic poly(vinyllactam(s)) is (are) used in an amount ranging from 0.1% to 10% by weight relative to the total weight of the composition.

15. The composition as claimed in any one of the preceding claims, characterized in that the fixing polymer is an anionic polymer chosen from:
polymers comprising carboxylic units derived from unsaturated monocarboxylic or dicarboxylic acid monomers of formula: 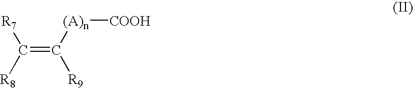
in which n is an integer from 0 to 10, A denotes a methylene group, optionally connected to the carbon atom of the unsaturated group, or to the neighboring methylene group when n is greater than 1, via a hetero atom such as oxygen or sulfur, R₇ denotes a hydrogen atom or a phenyl or benzyl group, R₈ denotes a hydrogen atom or a lower alkyl or carboxyl group, R₉ denotes a hydrogen atom, a lower alkyl group or a —CH₂—COOH, phenyl or benzyl group;
polymers comprising units derived from sulfonic acid, such as vinylsulfonic, styrenesulfonic or acrylamidoalkylsulfonic units.

16. The composition as claimed in any one of claims 1 to 14, characterized in that the fixing polymer is an amphoteric polymer chosen from polymers comprising units derived from:
a) at least one monomer chosen from acrylamides and methacrylamides substituted on the nitrogen with an alkyl radical,
b) at least one acidic comonomer containing one or more reactive carboxylic groups, and
c) at least one basic comonomer such as esters containing primary, secondary, tertiary and quaternary amine substituents of acrylic and methacrylic acids and the product of quaternization of dimethylaminoethyl methacrylate with dimethyl or diethyl sulfate.

17. The composition as claimed in any one of claims 1 to 14, characterized in that the fixing polymer is a nonionic polymer chosen from:
polyalkyloxazolines;
vinyl acetate homopolymers;
copolymers of vinyl acetate and of acrylic ester;
copolymers of vinyl acetate and of ethylene;
copolymers of vinyl acetate and of maleic ester;
copolymers of polyethylene and of maleic anhydride;
alkyl acrylate homopolymers and alkyl methacrylate homopolymers;
acrylic ester copolymers such as, for example, copolymers of alkyl acrylates and of alkyl methacrylates;
copolymers of acrylonitrile and of a nonionic monomer chosen, for example, from butadiene and alkyl (meth)acrylates;
copolymers of alkyl acrylate and of urethane; and
polyamides.

18. The composition as claimed in any one of claims 1 to 14, characterized in that the fixing polymer is a functionalized or nonfunctionalized, silicone or nonsilicone polyurethane.

19. The composition as claimed in any one of claims 1 to 14, characterized in that the fixing polymer is a polymer of grafted silicone type comprising a polysiloxane portion and a portion consisting of a nonsilicone organic chain, one of the two portions constituting the main chain of the polymer, the other being grafted onto said main chain.

20. The composition as claimed in any one of the preceding claims, characterized in that the fixing polymer is present in the composition at a relative weight concentration of between 0.1% and 10% and preferably between 0.5% and 5%.

21. The composition as claimed in any one of the preceding claims, characterized in that the cosmetically acceptable medium preferably consists of water or of one or more cosmetically acceptable solvents such as alcohols or water/solvent(s) mixtures, these solvents preferably being C₁-C₄ alcohols.

22. The composition as claimed in any one of the preceding claims, characterized in that it contains at least one additive chosen from anionic, cationic, nonionic and amphoteric surfactants, fragrances, screening agents, preserving agents, proteins, vitamins, provitamins, polymers other than those of the invention, plant, mineral or synthetic oils, polyols, for instance glycols or glycerol, silicones, fatty alcohols and any other additive conventionally used in cosmetic compositions.

23. A cosmetic haircare process, characterized in that it comprises the application of a composition as claimed in any one of the preceding claims.

24. The use of a composition as claimed in any one of claims 1 to 22 to fix and/or shape the hairstyle.
